(19) United States
(12) Patent Application Publication
(10) Pub. No.: US 2022/0193292 A1
(43) Pub. Date: Jun. 23, 2022

(54) SANITIZER SYSTEM

(71) Applicants: Silvia Elena Donahue, Agoura Hills, CA (US); Garret Joel Donahue, Agoura Hills, CA (US)

(72) Inventors: Silvia Elena Donahue, Agoura Hills, CA (US); Garret Joel Donahue, Agoura Hills, CA (US)

(21) Appl. No.: 17/128,333

(22) Filed: Dec. 21, 2020

(51) Int. Cl. A61L 2/24; A61L 2/10

(52) U.S. Cl. CPC A61L 2/24; A61L 2/10; A61L 2202/14; A61L 2202/121; A61L 2202/11; A61L 2202/122

(57) ABSTRACT

A sanitizer system includes a duct like enclosure with roll up doors at an entrance and/or an exit, the system for treating reusable items with one or more of ultraviolent light, an electrostatic fog, an ultra-low capacity spray, and a pressurized solution such as a pressurized water wash.

### BACKGROUND OF THE INVENTION

### Field of the Invention

The present invention relates to an article of manufacture for sanitizing reusable items such as shopping baskets and shopping carts. In particular, an enclosure for holding reusable items includes one or both contact and non-contact facilities for treatment of item surfaces.

### Discussion of the Related Art

Ultraviolet light (UV) sanitizing chambers for shopping carts are described in Taylor et al. (U.S. Pat. No. 7,791,044), Jones (US 2007/0012340), and Couvillion (US 2006/0186358). In each of these, means for directing UV light (i.e. reflectors) are described.

UV light suited to eradication of disease causing microorganisms includes UV-C light with a wavelength of 100 to 280 nanometers (nm), for example a light source comprising a wavelength of 254 nm. Notably, some studies indicate that reflected UV-C light suffers severe attenuation.¹ Other studies indicate that UV-C light does not reflect from any surface.² ¹ Elsevier ScienceDirect, Burns 46 (2020) 842-849. Ultraviolet-C decontamination of a hospital room: Amount of UV light needed.² germfalconwordpress.com/2017/01/19/uv-c-how-it-works/Reviewed Nov. 29, 2020.

### SUMMARY OF THE INVENTION

In the present invention a sanitizer system includes an enclosure with one or more means for treatment to eradicate microorganisms.

A first embodiment is a sanitizing system comprising: an enclosure providing a duct with entry and exit doors; a cart to be sanitized located within a central internal volume of the enclosure; opposed enclosure sidewalls and UV-C lamps for projecting UV light from each sidewall toward the central internal volume of the enclosure; an enclosure top and an enclosure bottom, UV-C lamps for projecting UV light from the top toward the central internal volume of the enclosure; an occupancy sensor for sensing the presence of life or prohibited items in the enclosure; a controller for automatically controlling entry and exit door position and door position sensors for sensing the position of the doors; and, an operator control panel including a keypad for code entry for enabling sanitizer operation, an emergency stop selector, a load cycle selector, a close cycle selector, and a sanitize cycle selector.

The embodiment may include at least one of the UV-C lamps moves with respect to the cart during the sanitize cycle.

The embodiment may include: an inactive state wherein the sanitizer automatically closes the entry and exit doors and deprives the UV lamps of power; an active state following the inactive state, the active state entered when the sanitizer recognizes a code entered on the keypad as a valid code to enable sanitizer operation; a load cycle entered only from the active state; within the load cycle, the entry door automatically opened and the exit door automatically closed if open; and, within the load cycle, entry and exit door positions sensed by the door position sensors wherein entry door motion stops when the door is fully open and exit door motion stops when the door is fully closed.

The embodiment may include: a close cycle entered only following a completed load cycle; within the close cycle, the entry door automatically closed only if the occupancy sensor indicates no life or prohibited items within the enclosure; and, if life or prohibited items are sensed within the enclosure, the entry and exit doors opened afterwhich the sanitizer enters the inactive state.

The embodiment may include: if no life or prohibited items are sensed, within the close cycle the entry door moves to close until the position sensed by the entry door position sensor indicates the the door is fully closed;

a sanitize cycle entered only following a completed close cycle; within the sanitize cycle, the UV lamps automatically illuminated to sanitize the enclosure contents; and, upon completion of the sanitize cycle the exit door is automatically opened to allow removal of the enclosure contents.

A second embodiment of the sanitizer system comprising: a circular enclosure having a side opening configured to accept carts such as those used by grocery stores and home improvement stores; an enclosure sidewall with internal UV-C lamps for projecting UV light; a turntable surrounded or partially surrounded by the enclosure; walls emanating from a turntable center that create spaces atop the turntable; the sanitizer for using the UV-C lamps to treat carts within the spaces; when the sanitizer is operating, direct light from the UV-C lamps prevented from reaching the opening by one of a) coordination of turntable movement with UV-C on/off light operation, and b) sanitizer geometry; and, an occupancy sensor for sensing the presence of life or prohibited items in the enclosure.

The embodiment may include: UV-C lamps mounted on at least two surfaces of the walls; and, electric power conducted from a stationary source to the wall mounted UV-C lamps via slip rings.

The embodiment may include: the turntable automatically locates carts at the opening such that treated carts can be removed and untreated carts can be reinserted.

The embodiment may include: cart bugs with cart bug indicators for informing cart users if a particular cart was already used after an immediately prior sanitizing treatment.

The embodiment may include: a communicator for informing store employees of the treated cart population.

### BRIEF DESCRIPTION OF THE DRAWINGS

The present invention is described with reference to the accompanying figures. These figures, incorporated herein and forming part of the specification, illustrate the invention and, together with the description, further serve to explain its principles enabling a person skilled in the relevant art to make and use the invention.

FIGS. 1A-B show a sanitizer system of the present invention.

FIG. 2 shows categories of components of the sanitizer system of FIGS. 1A-B.

FIG. 3A shows schematically an exploded view of the sanitizer system of FIGS. 1A-B.

FIG. 3B shows schematically an alternative arrangement of the UV tubes of the sanitizer system of FIGS. 1A-B.

FIG. 4 shows components of the sanitizer system of FIGS. 1A-B.

FIG. 5 shows an operator control panel for use with the sanitizer system of FIGS. 1A-B.

FIGS. 6A-B, 7A-B, 8A-B, 9A-B show operations the sanitizer system of FIGS. 1A-B may perform.

FIGS. 10A-C show first alternative arrangements of sanitizer systems.

FIGS. 11A-C show second alternative arrangements of sanitizer systems.

### DETAILED DESCRIPTION OF THE PREFERRED EMBODIMENTS

The disclosure provided in the following pages describes examples of some embodiments of the invention. The designs, figures, and description are non-limiting examples of embodiments they disclose. For example, other embodiments of the disclosed device and/or method may or may not include all of the features described herein. Moreover, disclosed advantages and benefits may apply to only certain embodiments of the invention and should not be used to limit the disclosed invention.

To the extent parts, components and functions of the described invention exchange electric power or signals, the associated interconnections and couplings may be direct or indirect unless explicitly described as being limited to one or the other. Notably, parts that are connected or coupled may be indirectly connected and may have interposed devices including devices known to persons of ordinary skill in the art.

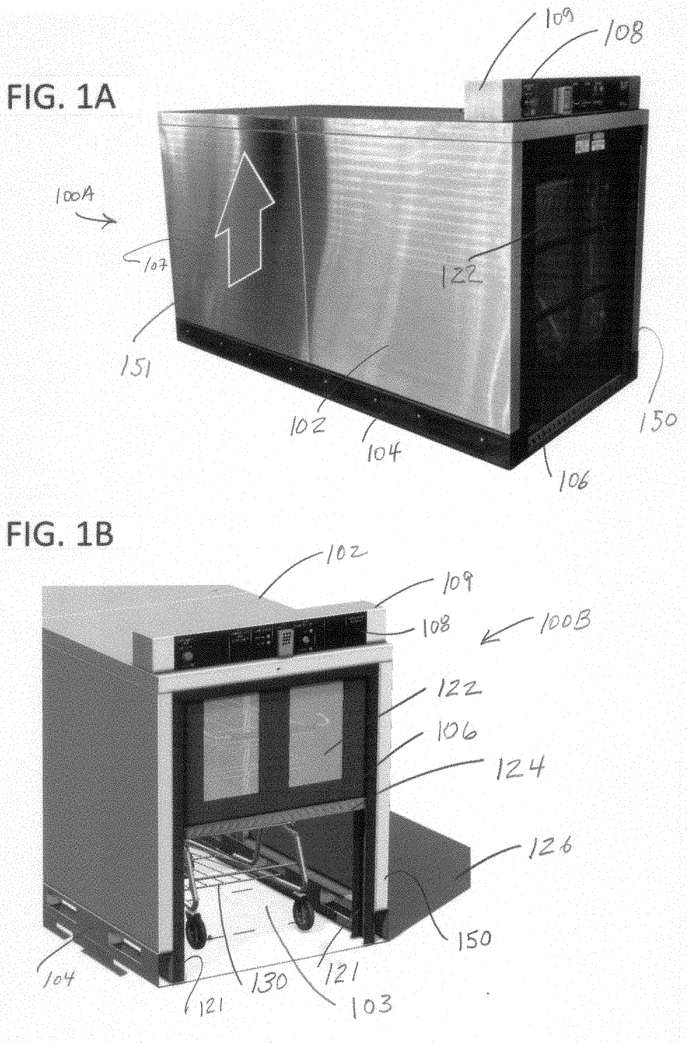

FIG. 1A shows a sanitizer system 100A. As seen, an enclosure 102 such as a metal enclosure having a base such as a rectangular base 104 provides a duct like structure having an entrance end 150 and an exit end 151. At the entrance end is a closed door 106. Various types of doors may be used, for example a hinged door, a split door, a vertically split door, a roll up door, and the like. Further, various door actuators may be used, for example electrically powered devices, air powered devices, hydraulic powered devices, and the like.

In some embodiments a door such as a roll up door 106 is used. This may be a roll up door using a segmented or flexible member such as a plastic member that retracts to expose the door opening. The door may have transparent portions 122 enabling a system operator to see inside the sanitizer while the door is closed. In various embodiments, an exit door 107 similar to the entrance door is provided at the exit end. Atop the enclosure and near the entrance end of the enclosure is an operator control panel 108 which may be mounted to an operator control panel box 109.

FIG. 1B shows a front end of the sanitizer system 100B. As seen, the sanitizer 100A is located adjacent to a curb 126. Here, the roll up door 106 at the entrance end 150 is shown partially open. The bottom of the roll up door may include a trim piece or seal 124 and at the vertical edges of the roll up door tracks 121 may be affixed to the enclosure to guide the door. In this view, a portion of the enclosure interior 103 is visible. Also visible is a portion of a grocery cart 130 positioned within the enclosure.

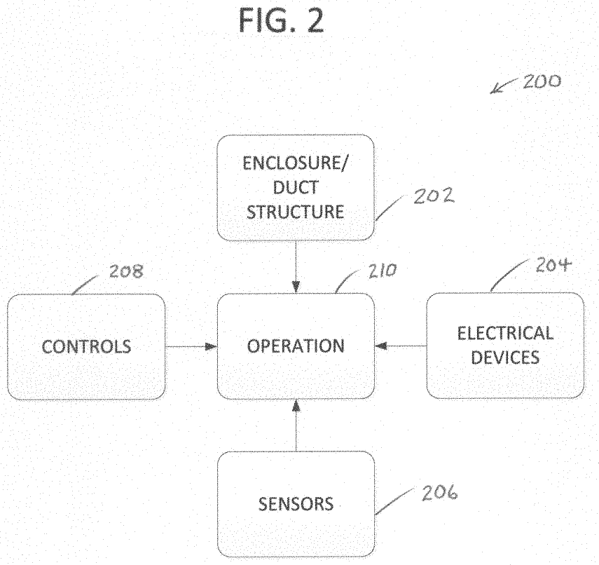

FIG. 2 shows components used during operation of the sanitizer system 200. An enclosure/duct structure 202 for containing reusable items such as shopping carts is equipped with electrical devices 204 such as drives, actuators, and/or motors for moving the roll up doors, sensors 206 such as door movement/position sensors, and controls 208 such as logic controls for opening and closing the doors. Interconnection of these components enables operation 210 of the sanitizer system that is safe and effective. Descriptions of each of the components follow. Note that while the descriptions may contemplate sanitization of a shopping cart, any reusable item compatible with the sanitizer system may be treated. As indicated by the context of use, where indicated treated means sanitized and where indicated untreated means the item/cart was used after the last sanitization.

Enclosure

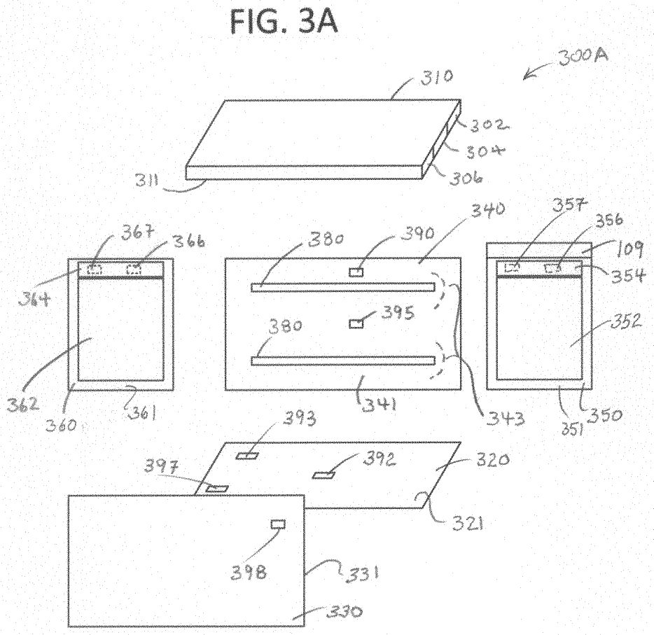

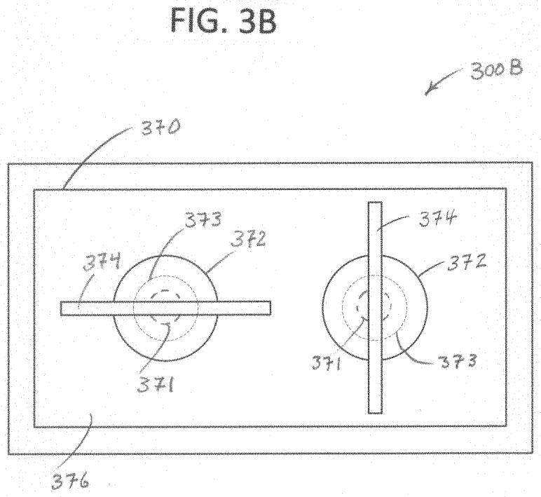

FIG. 3A shows an exploded schematic of the enclosure 300A. The enclosure includes a top 310, bottom 320, front side 330, back side 340, entrance end 350, and exit end 360. Equipment within the enclosure is described below. It should be noted that while this description associates the equipment with a particular part of the enclosure, the equipment may be rearranged, reoriented, or duplicated elsewhere in other embodiments. For example, a horizontal UV lamp on the back wall may be relocated or duplicated on the front wall. For example, a horizontal UV lamp may be reoriented to extend vertically. One or more carts may be nested (pushed together) or not within the enclosure.

Because UV-C light is harmful to skin and eyes, the sanitizer doors comprise a particular material such as a translucent or transparent material that blocks harmful UV spectrum light while allowing safe viewing of the enclosure 300A contents. Should the doors be raised during sanitizer 100A operation or sanitizer UV lamp 380 operation, door sensors deactivate the UV lamps as is further explained below.

The back side 340 is a vertical wall with an interior face 341. The back side interior face may include or be near to one or more UV lamps 380 (two shown) extending horizontally along the wall. In some embodiments, the lamp(s) are positioned within wall pockets 343 to avoid impact such as impact from a grocery cart 130 being inserted or removed from the sanitizer system. Sensors on the back wall 390 may include a sensor to detect enclosure contents, occupancy, or item insertion. Sensors on the back wall may include a moisture sensor 395. Contents detected may be any of a human, animal, object for limited treatment, or object that cannot be treated. The sensor or sensors may include sensing of any one or more of life, motion, sound, echoes, or size. Sensor technologies may include any of infrared, ultrasonic, sonic, microwave, photocell, light dependent resistor (“LDR”), thermal, and the like.

The front side 330 is a vertical wall with an interior face 331. Equipment on or near the front interior face may be a duplicate of or an alternative to any of the equipment located on the back face. An audible alarm and or light 398 may be located on this wall or on another wall of the enclosure.

The bottom 320 presents a horizontal surface with an interior face 321. Equipment on or near the bottom interior face may be a duplicate of or an alternative to any of the equipment located on the back face. The bottom face may include a weight sensor 392 for sensing the presence of enclosure contents. Weight or load cell sensor technologies may include any of strain gauges, pressure gauges, capacitance devices, and the like. The bottom face may include an insertion sensor 393 to sense full and/or proper insertion off a grocery cart such as a grocery cart nearest the exit door. The bottom face may include a pop-up cart stop 397 that is raised up to stop the cart during cart insertion and lowered down to allow passage of the cart via the exit door during cart removal.

The entrance end 350 is a vertical assembly with an interior face 351. Equipment in or near this assembly may include a door such as a roll up door 352 extending from an upper hood 354. In some embodiments, the door may be motorized and/or automatic. For example, a roll up door may include a hood motor/actuator 356 for lifting and lowering the door. One or more door sensors 357 may sense door motion, door position, door lifting limit and door lowering limit. Above the entrance door assembly is an operator control panel box 109 which may be mounted to the top 310 of the enclosure.

The exit end 360 is a vertical assembly with an interior face 361. Equipment in or near this assembly may include a door such as a roll up door 362 extending from an upper hood 364. In some embodiments, the door is motorized and/or automatic. For example, a roll up door may include a hood motor/actuator 366 for lifting and lowering the door. One or more door sensors 367 may sense door motion, door position, door lifting limit and door lowering limit.

The top 310 presents a horizontal surface with an interior face 311. Equipment on or near the bottom face interior surface may be a duplicate of or an alternative to any of the equipment located on the back face. In some embodiments, the top supports one or more of an electrostatic spray/fog unit 302, an ultra-low capacity spray unit 304, and a power washer 306. In other embodiments, any of these units may be supported by another part or wall of the enclosure. For example, the electrostatic spray unit and/or ultra-low capacity spray unit may be mounted to the front 330 and/or rear 340 wall.

FIG. 3B shows another implementation with moving or rotating UV lamps 300B. Here, the back wall 340 may include an inset portion 370 with an inset back wall 376. One or more motors 371 supported on or near a back face wall may rotate respective turntables 372. Affixed to these turntables are UV lamps 374. The UV lamps may be electrically powered by slip rings on or near the turntable such as back side slip rings of the turntable 373. Where two or more turntables are used, the related driving motors may be spaced or overlapping and synchronized to avoid contact of one rotating UV lamp with an adjacent UV lamp.

The arrangement of turntables and UV lamps described above in connection with the back wall 340 may be repeated or provided in the alternative on/near the front wall 330, on/near the bottom wall 320, or on/near the top wall 310.

Moving UV lamps may offer several advantages over fixed UV lamps. For example, one lamp illuminates at least a circular area with a diameter equal to the length of the UV lamp. For example, one lamp may, while rotating, pass proximate to the facing horizontal and vertical item/cart surfaces for treatment. For example, fewer lamps may be required to obtain the desired treatment results. These advantages and others may be obtained without reliance on or use of reflectors and reflected UV light emanating from the lamp(s).

Electrical and Controls

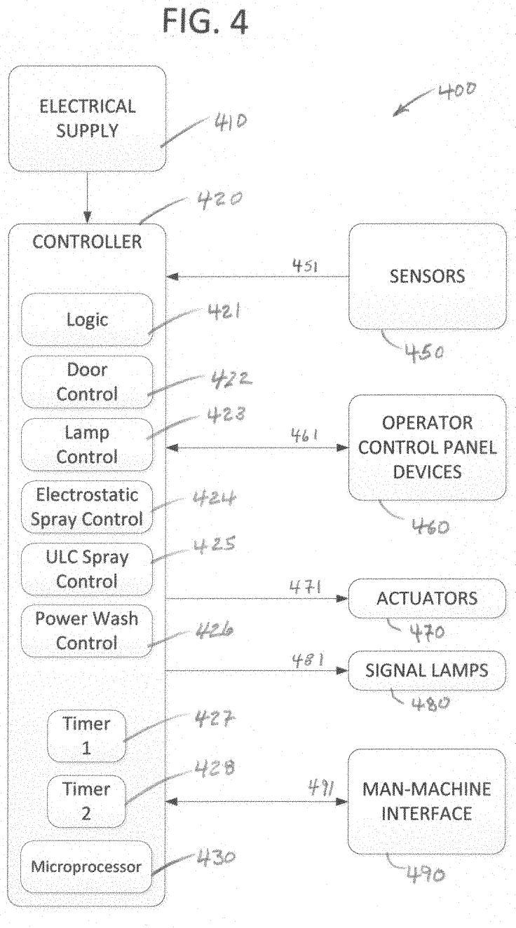

FIG. 4 shows electrical and control components in a block diagram 400. An electrical supply 410 provides electric power to a controller block 420 which may implement at least some controls using a microprocessor 430. Sensors 450, operator control panel devices 460, and an optional man-machine interface 490 may send signals 451, 461, 491 to the controller. Actuators 470, signal lamps 480, the operator control panel 460, and the man-machine interface 490 may receive signals 471, 481, 491 from the controller.

Sensors may include any of one or more of an entry door 350 sensor 357, exit door 360 sensor 367, an occupancy sensor 390, a cart insertion sensor 393, a weight sensor 392, and a moisture sensor 395. The sensor or sensor package 357, 367 associated with each door may be used to sense door motion and/or door position. Door sensors may also indicate the door fully up and the door fully down positions, for example using limit switches.

Actuators may include any of an actuator for opening/closing the entry door 356, an actuator for opening/closing the exit door 366, a UV lamp actuator, an electrostatic fog actuator, an ultra-low capacity spray actuator, and a power wash actuator. Operation of the entry and exit doors 350, 360 may be by a linear actuator or a rotary actuator (e.g., motor) such as an alternating current, direct current, servo, or stepper device.

Some embodiments include a man-machine interface 490 such as a man-machine interface with a video display. This interface may be used to monitor sanitation system status and performance using information received from the controller 420. This interface may be used to send instructions or commands to the controller including those associated with the operator control panel as explained below. For example, commands may determine the type of treatment (UV, Electrostatic Fog, Ultra Low Capacity Spray, Power Wash), load, start, and emergency stop. In addition, this interface may be used to display warnings and error conditions.

The controller 420 coordinates operations of sanitation system 100A. This includes coordination of the sanitation system components described above. Controller functions include one or more of door control 422, lamp control 423, electrostatic spray control 424, ultra-low capacity spray control 425, power wash control 426, various timers 427, 428, and logic 421 that integrates these functions.

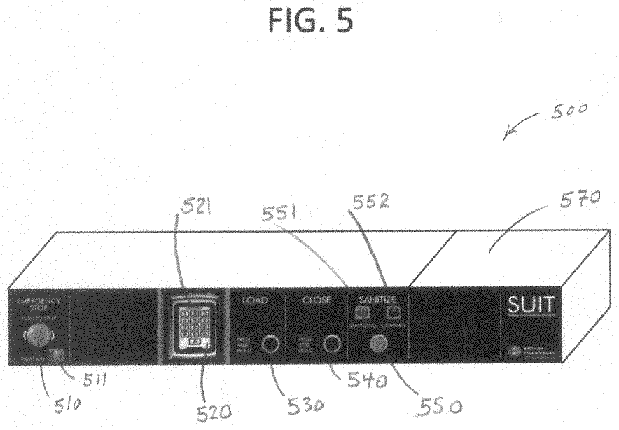

FIG. 5 shows an operator control panel 500 that may be included with the sanitizer system. The panel may include any of a keypad 521, an emergency stop button 510, a load button 530, a close button 540, and a sanitize button/selector 550. Some embodiments of the sanitizer system controller 420 implement at least some of the control functions in a microprocessor 430 such as a microprocessor located in the operator control panel 570.

In some embodiments, the emergency stop button 510 is used to rapidly remove power to any energized motors, lights, or controls in the case of an emergency. The emergency stop button may be used to power off the machine during periods of inactivity and immediately lockout the controls from unauthorized use. An indicator lamp 511 may show the status of the emergency button. Note that pressing the emergency stop button does not adversely affect the sanitizer in any manner and that twisting the button resets the switch.

In some embodiments, the entrance and exit doors automatically close when the sanitizer system 100A is unattended for long periods of time, for example using a 15, 30, or 60 minute timer 428. This feature is disabled if the emergency stop button is depressed.

The keypad 520 grants access to other sanitizer system controls. Entry of a code on the keypad can unlock any of the load 530, close 540, and sanitize 550 controls.

In some embodiments, sanitizing a load begins with loading the enclosure 300A. Once loaded, the entrance and exit doors 350, 360 are closed and treatment of the load can begin. After treatment, the exit door 360 is opened and the load is removed. This process may be repeated as needed.

Enable Operation

Treating a first load may follow a control lockout after a power loss, emergency stop 510, or five minutes of timed inactivity 427. To enable the controls, an operator must enter a particular code using the keypad 520. A successful code entry may cause a keypad indicator 521 such as a green light to turn on. Once enabled, the load 530, close 540, and sanitize 550 buttons/selectors will respond normally such that a first load and subsequent loads can be treated. Indicators 551, 552 such as blue and green lamps may show sanitizing is in progress and sanitizing is complete. As noted, five minutes of inactivity locks the controls after which the operator must reenter the code via the keypad.

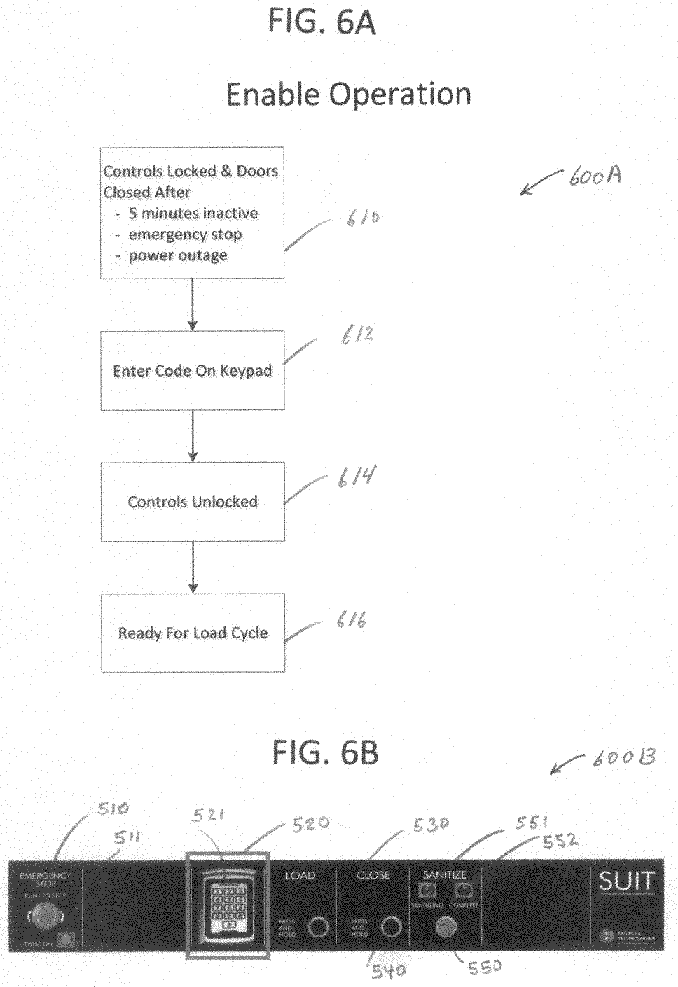

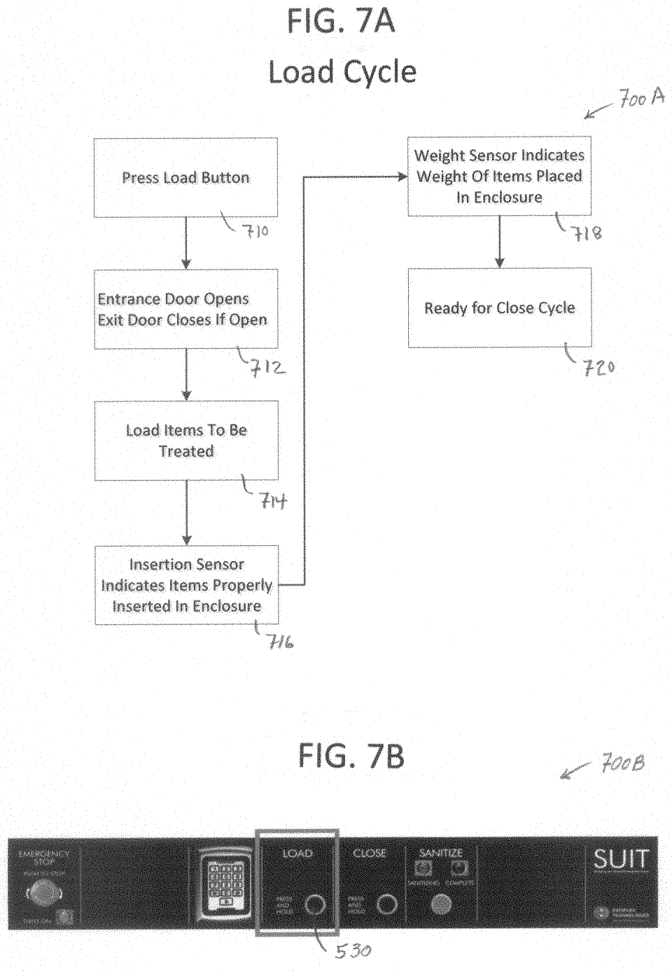

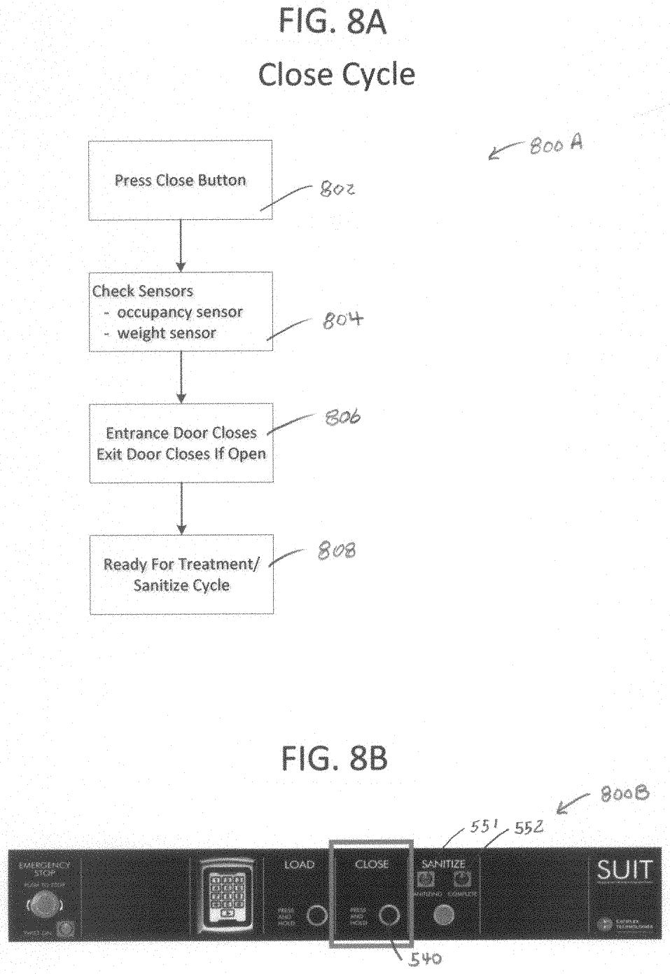

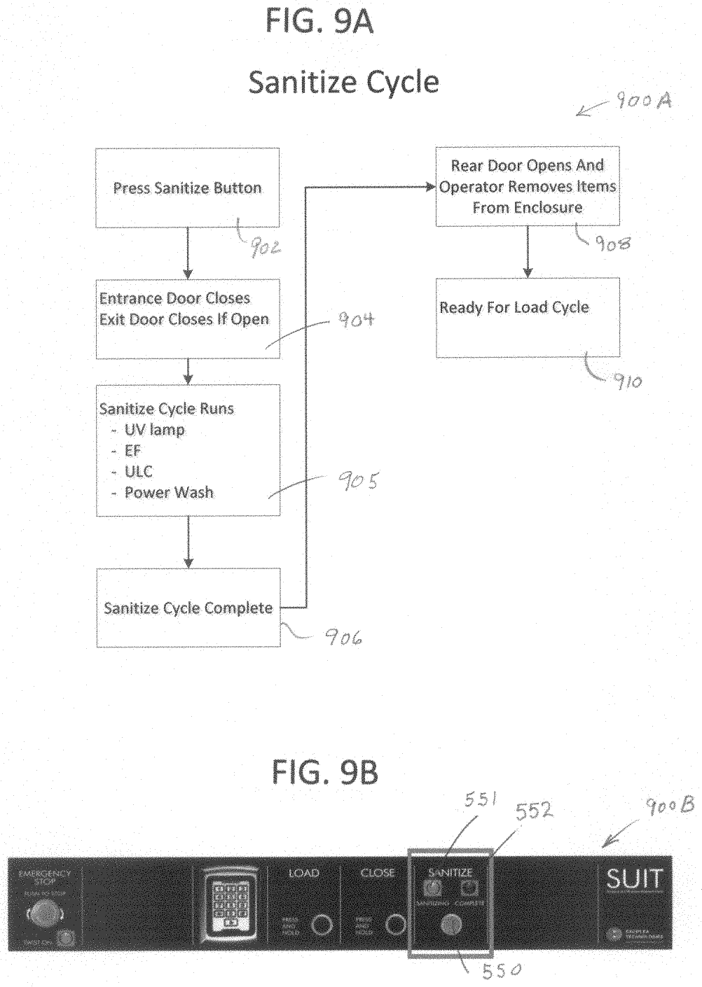

FIGS. 6A-B show an example 600A-B of enabling sanitizer system operation. At a first step 610, the controls 530, 540, 550 are locked and the entrance and exit doors 350, 360 are closed. This state follows a period of five minutes of inactivity, an emergency stop, or a power outage. In a second step 612, an operator uses the keypad 520 to enter a code which unlocks the controls 614. At this point, the sanitizer system is ready for the load cycle 616.

Load Cycle

FIGS. 7A-B show an example 700A-B of a sanitizer loading cycle. In a first step 710, the operator presses the load button 530 which in a second step 712 opens the entrance door 350 and closes the exit door 360 if it is open. Where door sensors 357, 367 include limit switches or a similar means of providing a stop signal, the operator may depress the load button until the doors have automatically stopped moving, for example due to contact with a limit switch. When the doors have stopped moving, the operator loads the items 714 to be treated into the sanitizer enclosure 300A. In some embodiments, a pop-up stop 397 is used to limit travel of the items/cart into the enclosure and/or an insertion sensor 393 may indicate that an item is fully inserted 716. And in some embodiments a weight sensor 392 may indicate the weight of the inserted item, for example to show item insertion 718. Following loading, the sanitizer is ready for the close cycle 720.

FIGS. 8A-B show an example 800A-B of a sanitizer close cycle. In a first step 802, the operator presses the close button 540. Before the controls act to close sanitizer doors that are open 350, 360, indications from the occupancy sensor 390 and/or weight sensor 392 are evaluated 804. If the occupancy and/or weight sensor indicates that life or a prohibited item is within the sanitizer enclosure 300A, the sanitizer system entrance door 350 will not close and a warning will alert the operator. Warnings include blinking of both sanitize lamps 551, 552 and/or an audible/visible warning 398. In some embodiments, the enclosure must be inspected and cleared before the entrance door can be closed.

When the close button 540 is pressed and the sensors 390, 392 indicate no life or prohibited items are within the enclosure, in a third step 806 the entrance door 350 closes and the exit door 360 closes if it is open. In a fourth step 808 the sanitizer system 100A is now ready to treat/sanitize the items within.

Sanitize Cycle

FIGS. 9A-B show an example 900A-B of a sanitize cycle. In a first step 902, the operator selects the sanitize setting 550 which is a selector switch, a button, or a combination of similar features. In the case that multiple treatments are offered such as UV, EF, ULC, and/or pressure wash, the sanitize setting selects which one or which combination of these treatments is to be performed. In a second step 904, the entry door 350 closes and the exit door 360 closes if it is open. Once the sanitize cycle begins, in a third step 905 a lamp 551 may indicate the cycle is in progress. It should be noted that in some embodiments, the entrance and exit doors 350, 360 cannot be opened during the sanitize cycle.

At the end of the sanitize cycle 906, a cycle end lamp 552 may illuminate. At the end of the sanitize cycle 908, the exit door 360 opens and the pop up stop 393, if present, retracts. The items/carts 130 may now be removed from the sanitizer enclosure 300A and load/close/sanitize cycles repeated to treat another load if desired 910.

Cycles including those mentioned above and including manual cycles and automated cycles 1) enable, 2) load, 3) close, 4) sanitize, 5) repeat may be configured to suit particular applications. For example, the sequence 1/2/3/4/5 may initiate use of the sanitizer and be followed by 2/3/4/5 for subsequent loads. For example, 3 may require that 2 precede it. For example, 4 may require that 3 precede it. For example 5 may require that 4 precede it. In some embodiments 2/3/4/5 must be performed in sequence. In some embodiments intermediate steps are performed between cycles. In some embodiments no intermediate steps are performed between cycles.

The description above provides, inter alia, a sanitizer system with a single entry and a single exit, the carts to be sanitized being loaded into the entrance and thereafter extracted from the exit. Here, carts are moved along or substantially along a single straight line since there is only one sanitizer system. Embodiments of the sanitizer system described below may utilize different cart and sanitizer arrangements. For example some arrangements may allow for a gap between each cart during treatment and/or provide multiple cart paths through multiple treatment zones in one or more sanitizer systems. Further, the sanitizer system above and those described below may utilize a mechanism to move the carts into and/or out of a sanitizer system. It should also be noted that where suitable, elements of the sanitizer system above may be repeated in the embodiments below. For example, the above-described lamps and controls may be repeated in a similar fashion in the sanitizer systems below.

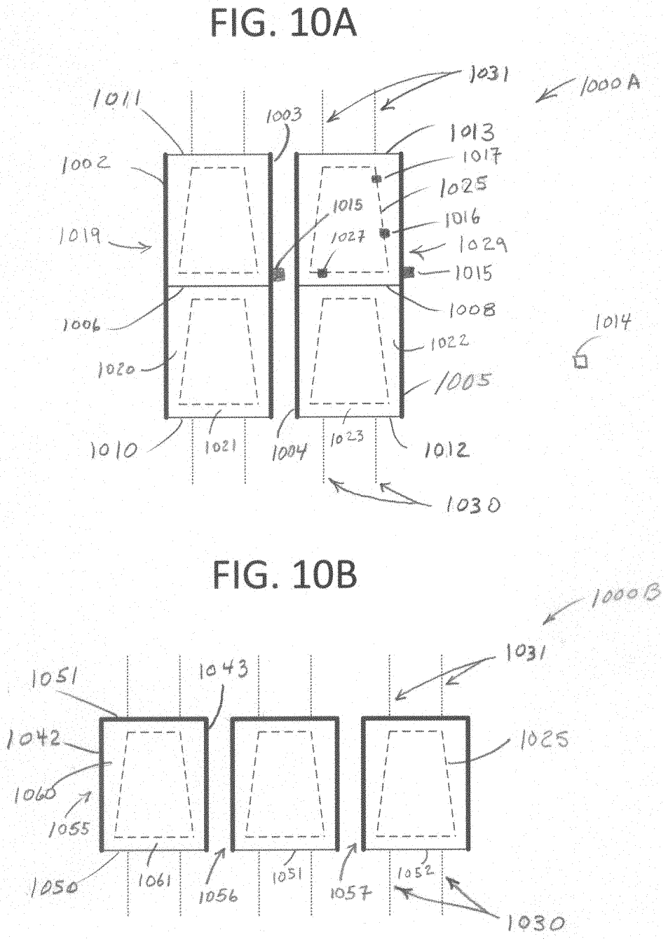

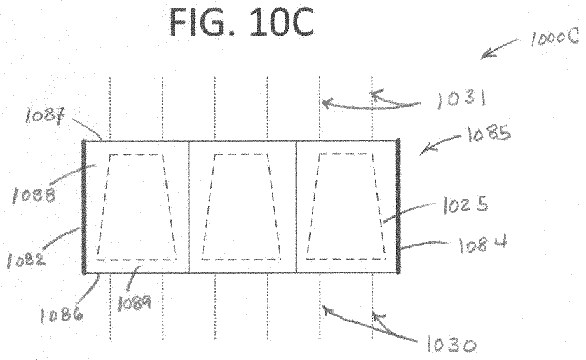

FIG. 10A shows a first schematic overhead view of carts within sanitizer systems 1000A. In the figure a first sanitizer system 1019 is shown adjacent to a second sanitizer system 1025 in a modular/separable configuration. Here, multiple stand-alone sanitizer systems are used to increase the capacity of a sanitizer installation. Two or more sanitizer systems may be used in such an installation.

The first sanitizer system enclosure 1019 has opposed vertical walls 1002, 1003 and a top 1020 opposite a floor 1021 creating a duct or chute in which items such as carts 1025 are treated. An entry door 1010 is opposite an exit door 1011 and carts 1025 are located therebetween during treatment. Note that here, two carts are located in the enclosure and that the carts are spaced apart. A cart locator, spacer or additional treatment station 1006 may be located between carts.

The second sanitizer system enclosure 1029 has opposed vertical walls 1004, 1005 and a top 1022 opposite a floor 1023 creating a duct or chute in which items such as carts 1025 are treated. An entry door 1012 is opposite an exit door 1013 and carts 1025 are located therebetween during treatment. Note that here, two carts are located in the enclosure and that the carts are spaced apart. A cart locator, spacer or additional treatment station 1008 may be located between carts. In some embodiments, two sets of the controls and sensors described above are used with respective enclosures 1019, 1029.

Also shown in the figure is a mechanism for moving carts into 1030 and/or out of 1031 the respective enclosures 1019, 1029. For example, the mechanism may include one or more of tracks, cables, chains, ropes or the like. For example the mechanism may include one or more of electrically powered devices, air powered devices, hydraulic powered devices, and the like. For example, a cart conveyance may include a moving surface on which the cart sits or by which the cart is tugged.

Any of the sanitizer systems mentioned above or below may include smart cart features. For example, a cart user may operate a movable flag or indicator to show the cart has been used and a cart attendant may operate the movable flag or indicator to show the card has been sanitized and/or is ready for another cart user. Exemplary indicators include devices that once set to show use can only be reset to show sanitized with an appropriate code, key, or similar means.

In some embodiments these features include a sanitizer system communicator 1015 or communicators and/or cart bugs 1016 any of which may be distributed and/or multi-element devices. Devices implementing these features may utilize one or more of radios, RFID, WIFI, Infrared, Infrared RTLS, Bluetooth, optical badges, optical readers, signal transmission, signal reception, memory and/or processors for processing and holding cart utilization and other statistics, and the like. In an embodiment, the communicator records unique cart identifiers as the carts are treated such that treated carts leaving the sanitizer system are known. The communication may be located within range of the bugs, at the sanitizer system, or inside a store using the items/cart.

In some embodiments, a similarly identified bug on the treated card, such as a cart leaving the sanitizer system, receives a message from the communicator such as a communicator located at the sanitizer system which activates a bug indicator 1017 to show the cart is a treated cart. The bug indicator changes state to show/indicate the cart has been used after treatment when a bug detector 1027 senses the cart has been used after treatment. The indicator may be any suitable illuminator(s), display, annunciator, or the like. This detector may be any suitable switch, motion sensor, location sensor, noise sensor, or the like.

Some embodiments including one or more of the smart cart features above provide for informing cart attendants or others such as store employees of the population of treated carts, this population being indicative of the need to alter the rate of carts being treated. Some embodiments including one or more of the smart cart features above provide a computing device and/or display 1014 for receiving statistics from the communicator 1015 such as the population of treated carts, rate of carts being treated, rate of carts being used, carts that have not been treated for a particular period of time, and cart utilization for example cart utilization based on time between treatments.

FIG. 10B shows a second schematic overhead view of carts within sanitizer systems 1000B. In the figure a first sanitizer system enclosure 1056 between adjacent sanitizer system enclosures 1055, 1057 in a modular/separable configuration. Here, multiple stand-alone sanitizer systems are used to increase the capacity of a sanitizer installation. Two, three, or more sanitizer systems may be used in such an installation.

As shown, each of the enclosures 1055, 1056, 1057 holds a single cart. An exemplary enclosure has three adjoining vertical walls 1042, 1051, 1043 with a top 1060 and floor 1061 forming a duct or chute. Each of the enclosures has a single door 1050, 1051, 1052 for loading and unloading the cart before and after treatment.

Also shown in the figure is a mechanism for moving the cart 1025 into 1030 and/or out of 1031 the respective enclosures 1055, 1056 1057. For example, the mechanism may include one or more of tracks, cables, chains, ropes or the like. For example the mechanism may include one or more of electrically powered devices, air powered devices, hydraulic powered devices, and the like. For example, a cart conveyance may include a moving surface on which the cart sits or by which the cart is tugged.

FIG. 10C shows a third schematic overhead view of carts within a sanitizer system 1000C. In the figure a sanitizer system enclosure 1085 encloses three side by side carts 1025 in a non-modular/non-separable configuration. For example, a single sanitizer system may hold multiple side by side carts to increase the capacity of a sanitizer installation.

As shown, a single sanitizer enclosure 1085 holds three side by side carts. The enclosure has two opposing walls or sides 1082, 1084 with a top 1088 and a floor 1089 forming a duct or chute. An entrance door 1086 and an exit door 1087 extend between the sides. Carts are loaded into the enclosure via the entry door and carts are extracted from the enclosure via the exit door.

Also shown in the figure is a mechanism for moving the cart 1025 into 1030 and/or out of 1031 the respective enclosures 1055, 1056 1057. For example, the mechanism may include one or more of tracks, cables, chains, ropes or the like. For example the mechanism may include one or more of electrically powered devices, air powered devices, hydraulic powered devices, and the like. For example, a cart conveyance may include a moving surface on which the cart sits or by which the cart is tugged.

The descriptions above include, inter alia, sanitizer systems arranged in a linear fashion and may include a mechanism for moving the carts. Systems with similar features to those described above may not be arranged in a linear fashion. For example, the above-described lamps and portions of the sanitizer system controls may be repeated in a similar fashion in the sanitizer systems below which are not arranged in a linear fashion. For example, sanitizer systems may use curvilinear or circular cart paths.

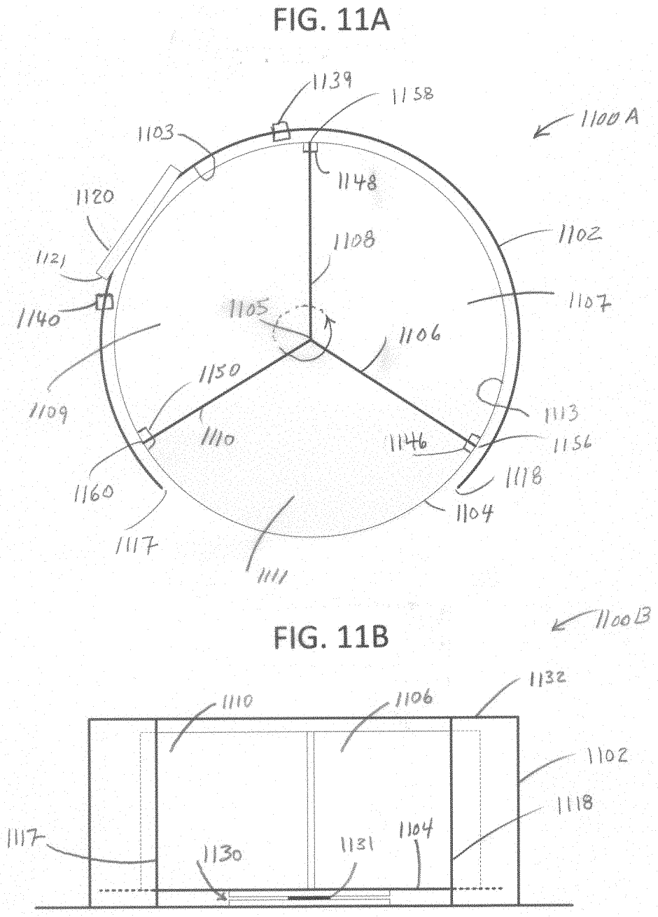

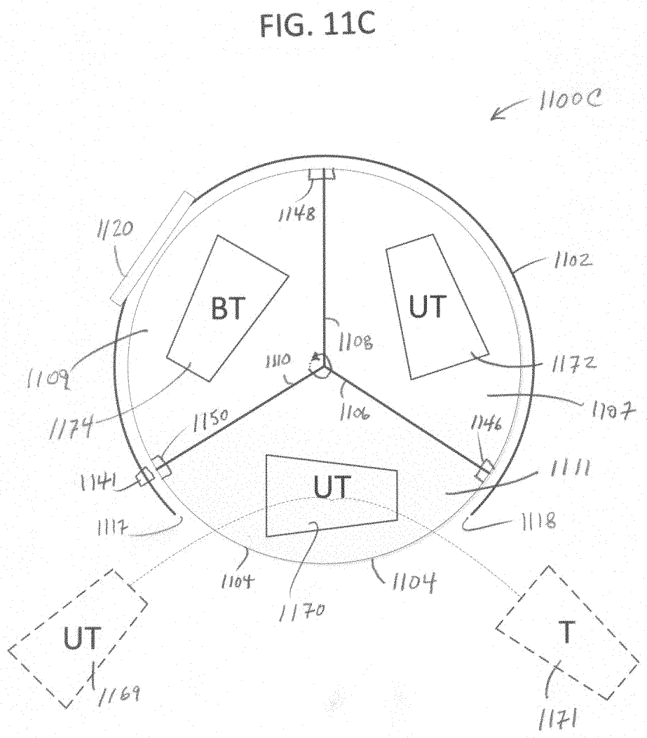

FIG. 11A shows a plan view of a circular sanitizer system arrangement 1100A. Here, a circular or semi-circular enclosure 1102 surrounds a turntable such as a circular turntable 1104. A top (see 1132 in FIG. 11B) may be included on the enclosure. A gap/entrance in the enclosure 1117, 1118 may provide for loading carts onto the turntable. Upstanding walls 1106, 1108, 1110 sitting on the turntable emanate from the center 1105 of the turntable. Spaces 1107, 1109, 1111 are created between the upstanding walls. As the turntable rotates, ultraviolent light(s)/lamp(s) similar to those described above 1120 are for treating carts as they pass by the lamps. Note that the lamps may be positioned across from the entrance such that light from the lamps does not or substantially does not reach the entrance due to the presence of one or more of the walls therebetween. See for example wall 1110 which is between the lamps and the entrance such that direct light from the lamps is blocked or substantially blocked. See for example walls 1108, 1106 which may block reflected light from reaching the entrance.

Notably, the enclosure wall ultraviolet lamp 1120 may be accompanied by one or more ultraviolet lamps on the turntable wall(s) 1106, 1108, 1110, floor, and/or top (not shown). Slip rings 1131 may be used to conduct electric power from a stationary source, through the slip rings, to the turntable 1104 and to the ultraviolet lamp(s). In other embodiments, additional turntables (3X) (not shown) in the floor of each space 1107, 1109, 1111 may be used to expose the cart to ultraviolet light emanating from a single source or direction.

Ultraviolet lamp 1120 operation may be by various means. For example, the ultraviolet lamp may run/operate whenever the sanitizer system 1100A is activated or whenever the turntable 1130 is activated. For example, the light may be turned on manually or automatically when a wall tip 1156, 1158, 1160 passes the ultraviolet light, for example at 1140. For example, the light may be turned off whenever a wall tip nears the ultraviolet light, for example at 1139. Wall tip sensors or sensor portions 1146, 1148, 1160 may cooperate with sensor or sensor portions before 1139 and after the 1140 the ultraviolet light to automatically turn the light off and to automatically turn the light on. Sensors may be any of electrical switches, electrical devices such as magnetic/electric field sensors, optical sensors, or the like. For example, fixed enclosure wall sensors such as 1139, 1140 operate as or with an electric switch that directly or indirectly provides electric power to the ultraviolet lamps. In some embodiments automated ultraviolet light controls ensure the ultraviolet light is not operated when any wall 1106, 1108, 1110 is between the entrance limits 1117, 1118. In some embodiments, the entrance limits are spaced such that irrespective of when the ultraviolet lamps operate, direct exposure to ultraviolet lamp light is prevented by one or more of the walls.

FIG. 11B shows an elevation view of the sanitizer system 1100B. Here, the turntable 1104 is shown atop a transport system 1130. The transport system may utilize a drive powered by electricity, hydraulics, air, or the like. For example the drive may be an electric motor such as a rotary motor with or without a gearbox.

FIG. 11C illustrates sanitizer system operation 1100C. Treated or to be treated carts are indicated as: UT for untreated; T for treated; and, BT for being treated.

As shown in the figure, a treated cart 1171 has been moved from the entrance 1117, 1118 and an untreated cart 1170 from a line or one or more untreated carts 1170, 1169 has taken its place on the turntable 1104 in space 1111. Ahead (counterclockwise) of cart 1170 is untreated cart 1172 in space 1107. In some embodiments space 1111 has no ultraviolet lamps (as shown) and in some embodiments space 1111 has ultraviolet lamps or another treatment mode such as electrostatic spray 302, low capacity spray 304 or power wash 306 in which case cart 1172 may be being treated.

In space 1109, a cart 1174 is being treated using ultraviolet lamps 1120 (as shown). When the cart(s) is treated, the turntable continues or restarts rotation moving counterclockwise to enable removal of treated cart 1174 from the sanitizer system and loading of another cart such as cart 1169.

Turntable 1104 operation may be manual or automatic such that the turntable locates an exposed space 1107, 1109, 1111 at the entrance 1117, 1118 and stops or moves an exposed space slowly at the entrance such that the treated cart can be removed and an untreated cart added. In an embodiment the sensors described above provide some or all of the features required manual or automatic operation. In an embodiment, a fixed sensor or sensor part 1141 near the entrance senses a nearby wall 1150 and stops or slows the turntable. In an embodiment, the turntable operates to treat a cart for a particular time and during the time the cart is exposed to treatment either stops or slows.

While various embodiments of the present invention have been described above, it should be understood that they have been presented by way of example only, and not limitation. It will be apparent to those skilled in the art that various changes in the form and details can be made without departing from the spirit and scope of the invention. As such, the breadth and scope of the present invention should not be limited by the above-described exemplary embodiments, but should be defined only in accordance with the following claims and equivalents thereof.

1. A sanitizing system comprising:
an enclosure providing a duct with entry and exit doors;
a cart to be sanitized located within a central internal volume of the enclosure;
opposed enclosure sidewalls and UV-C lamps for projecting UV light from each sidewall toward the central internal volume of the enclosure;
an enclosure top and an enclosure bottom, UV-C lamps for projecting UV light from the top toward the central internal volume of the enclosure;
an occupancy sensor for sensing the presence of life or prohibited items in the enclosure;
a controller for automatically controlling entry and exit door position and door position sensors for sensing the position of the doors; and,
an operator control panel including a keypad for code entry for enabling sanitizer operation, an emergency stop selector, a load cycle selector, a close cycle selector, and a sanitize cycle selector.

2. The sanitizer system of claim 1 wherein at least one of the UV-C lamps moves with respect to the cart during the sanitize cycle.

3. The sanitizer system of claim 1 further comprising:
an inactive state wherein the sanitizer automatically closes the entry and exit doors and deprives the UV lamps of power;
an active state following the inactive state, the active state entered when the sanitizer recognizes a code entered on the keypad as a valid code to enable sanitizer operation;
a load cycle entered only from the active state;
within the load cycle, the entry door automatically opened and the exit door automatically closed if open; and,
within the load cycle, entry and exit door positions sensed by the door position sensors wherein entry door motion stops when the door is fully open and exit door motion stops when the door is fully closed.

4. The sanitizer system of claim 3 wherein:
a close cycle entered only following a completed load cycle;
within the close cycle, the entry door automatically closed only if the occupancy sensor indicates no life or prohibited items within the enclosure; and,
if life or prohibited items are sensed within the enclosure, the entry and exit doors opened afterwhich the sanitizer enters the inactive state.

5. The sanitizer system of claim 4 wherein:
if no life or prohibited items are sensed, within the close cycle the entry door moves to close until the position sensed by the entry door position sensor indicates the the door is fully closed;
a sanitize cycle entered only following a completed close cycle;
within the sanitize cycle, the UV lamps automatically illuminated to sanitize the enclosure contents; and,
upon completion of the sanitize cycle the exit door is automatically opened to allow removal of the enclosure contents.

6. A sanitizing system comprising:
a circular enclosure having a side opening configured to accept carts such as those used by grocery stores and home improvement stores;
an enclosure sidewall with internal UV-C lamps for projecting UV light;
a turntable surrounded or partially surrounded by the enclosure;
walls emanating from a turntable center that create spaces atop the turntable;
the sanitizer for using the UV-C lamps to treat carts within the spaces;
when the sanitizer is operating, direct light from the UV-C lamps prevented from reaching the opening by one of a) coordination of turntable movement with UV-C on/off light operation, and b) sanitizer geometry; and,
an occupancy sensor for sensing the presence of life or prohibited items in the enclosure.

7. The sanitation system of claim 6 further comprising:
UV-C lamps mounted on at least two surfaces of the walls; and,
electric power conducted from a stationary source to the wall mounted UV-C lamps via slip rings.

8. The sanitizing system of claim 6 wherein the turntable automatically locates carts at the opening such that treated carts can be removed and untreated carts can be reinserted.

9. The sanitizing system of claim 8 further comprising:
cart bugs with cart bug indicators for informing cart users if a particular cart was already used after an immediately prior sanitizing treatment.

10. The sanitizing system of claim 9 further comprising:
a communicator for informing store employees of the treated cart population.
